(19) United States
(12) Patent Application Publication
(10) Pub. No.: US 2023/0190447 A1
(43) Pub. Date: Jun. 22, 2023

(54) IMPLANTABLE LENS CAPSULE FOR INTRAOCULAR LENS INSERTION

(71) Applicant: THE JOHNS HOPKINS UNIVERSITY, Baltimore, MD (US)

(72) Inventors: Albert Jun, Towson, MD (US); Angela Zhu, Baltimore, MD (US); Peter Gehlbach, Monkton, MD (US)

(21) Appl. No.: 18/167,546

(22) Filed: Feb. 10, 2023

Related U.S. Application Data
(63) Continuation of application No. 16/644,589, filed on Mar. 5, 2020, now abandoned.
(60) Provisional application No. 62/554,080, filed on Sep. 5, 2017.

(51) Int. Cl. A61F 2/16

(52) U.S. Cl. CPC A61F 2/16; A61F 2002/16901; A61F 2220/0016; A61F 2240/001; A61F 2220/0075

(57) ABSTRACT

An embodiment in accordance with the present invention provides a synthetic lens capsule for use in IOL fixation that is compatible with current and future IOL implants and relatively straightforward to implant. The device of the present invention can be inserted into the eye via a small (<3.0 mm) incision in the eye wall. The device of the present invention is compatible with all known or conceivable ways of securing the device to the eye. Securement is preferably done with at least two-point securement. In some embodiments, the device may also include a proprietary or conventional method of securement. To allow for expansion of the device as space permits, the device can also include a flexible ring structure. The device can include at least one aperture for the passage of light. The device of the present invention simplifies visual rehabilitation in cases of inadequate capsular support for IOL fixation.

### CROSS REFERENCE TO RELATED APPLICATION

This application is continuation of U.S. application Ser. No. 16/644,589 filed on Mar. 5, 2020, which is a 35 U.S.C. § 371 U.S. national entry of International Application PCT/US2018/049517, having an international filing date of Sep. 5, 2018, which claims the benefit of U.S. Provisional Application No. 62/554,080, filed Sep. 5, 2017, the content of each of the aforementioned applications is herein incorporated by reference in their entirety.

### FIELD OF THE INVENTION

The present invention relates generally to medical devices. More particularly, the present invention relates to an implantable lens capsule for intraocular lens insertion.

### BACKGROUND OF THE INVENTION

Cataract surgery is the most common intraocular surgery, but various circumstances may compromise the ability to safely insert an intraocular lens (IOL) implant within the native lens capsule or anterior to the native lens capsule in the ciliary sulcus after cataract extraction. Alternatives for IOL placement within the native lens capsule or ciliary sulcus such as anterior chamber IOLs, iris-fixated lenses, or scleral-fixated lenses have been described. These alternate methods require specialized adjustments in IOL design or surgical techniques and are associated with a range of intra- and post-operative complications.

Ideally, a single procedure is performed to both extract the native lens and place an intraocular lens (IOL) implant centered stably in the native lens capsule, which is the basement membrane of the lens epithelial cells. The lens capsule is supported by the zonular apparatus, which is a microfibril complex passing from the pars plana and ciliary body to the lens capsule. A variety of surgical circumstances and predisposing conditions can compromise the ability to safely insert an IOL in the native lens capsule during or after cataract extraction. When standard endocapsular IOL placement cannot be securely accomplished, surgical options exist to ensure IOL stability and optimal visual outcomes. In some cases of sub-total zonular dehiscence, a capsular tension ring (CTR) may be placed into the lens capsule prior to IOL insertion. The goal of a CTR is to distribute uneven forces more evenly throughout the lens capsule and remaining zonules to prevent IOL decentration. A posterior chamber IOL can also be placed in the ciliary sulcus (space between anterior lens capsule and iris) if sufficient anterior capsule and zonules remain intact. If endocapsular or sulcus IOL placement is no longer an option, alternative approaches include anterior chamber angle support (anterior chamber IOL), fixation to the iris with sutures or iris claw, and fixation to the sclera with or without sutures.

Alternatives for IOL placement in the absence of adequate capsular support all have significant shortcomings. Anterior chamber IOL (ACIOL) implants are in close proximity to the cornea and angle, increasing risk for pseudophakic bullous keratopathy, peripheral anterior synechiae formation, trabecular meshwork damage leading to glaucoma, and erosion of haptics into the iris or ciliary body leading to uveitis and hyphema. Iris-fixated lenses can lead to disruption of iris architecture and chronic iris trauma leading to intraocular inflammation, retinal edema, dyscoria, excess or inadequate pupillary dilation, and iridodialysis. Suture breakage can lead to IOL subluxation or dislocation. Scleral suture fixation of IOLs involves passing sutures through uveal tissue which increases risk of cystoid macular edema and vitreous hemorrhage. There is also potential for suture erosion resulting in a risk of endophthalmitis. Suture breakage leads to lens instability/decentration post-operatively. Sutureless scleral-fixated IOLs decrease the risk of suture related complications, but they require a high level of technical skill and still have risk of vitreoretinal complications related to the creation of sclerotomies. All current approaches for IOL fixation in the absence of adequate capsular support require significant alterations in surgical approaches and are subject to complications related to IOL positioning (decentration, subluxation, dislocation, or tilt), inflammation, or defects of the material used or the structure of the eye. These alternative techniques also involve intraoperative adjustments in pre-calculated IOL power, as anterior shifts in an IOL's position change its refractive effect.

In addition, all of the existing methods include adapting an IOL for placement in an eye without a capsule. This requires additional inventory of specialized IOLs for interventions in patients without adequate capsular support or changes to be made to off the shelf IOLs before insertion.

Therefore, it would be advantageous to provide an implantable lens capsule for intraocular lens insertion that is compatible with any standard IOL.

### SUMMARY OF THE INVENTION

The foregoing needs are met, to a great extent, by the present invention, wherein in one aspect a device for insertion into an eye includes a capsule having an outer surface defining an inner chamber configured to receive an intraocular lens (IOL). The capsule has an anterior aperture configured to allow the IOL to pass into the inner chamber. The device also includes an attachment mechanism for attaching the capsule to a sclera of the eye.

In accordance with an aspect of the present invention, the capsule is formed from an optically clear material or an opaque material. The capsule can be formed from a polymer. The device includes a flexible outer ring. The flexible outer ring can include a notch, discontinuity, or gap. The attachment mechanism can take the form of a suture. The suture can have a needle tip or a loop tip. The suture can also be a double armed suture. The device can include a posterior aperture. The device can be formed from a material having strength, stability, and biocompatibility. The device is compatible with IOLs configured to be used in a natural capsule.

In accordance with another aspect of the present invention, a method for providing a capsule for an intraocular lens (IOL) includes creating a synthetic lens capsule. The synthetic lens capsule includes an outer surface defining an inner chamber configured to receive the IOL. The capsule has an anterior aperture configured to allow the IOL to pass into the inner chamber. The method also includes providing an attachment mechanism for attaching the capsule to a sclera of the eye.

In accordance with yet another aspect of the present invention, the method includes surrounding the synthetic lens capsule with a flexible ring. The method includes forming the synthetic lens capsule from a biocompatible plastic. The method includes using a suture as the attachment mechanism. The method includes ending the suture with a needle tip or a loop tip. The method also includes using a single arm or a double arm suture.

Additionally, the method includes forming at least a portion of the capsule from an opaque material and using an opaque material that is compatible with a YAG laser.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a top down view of an implantable lens capsule device, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a synthetic lens capsule for use in IOL fixation that is compatible with current and future IOL implants and relatively straightforward to implant. Preferably, the lens capsule is formed from a material that includes strength, stability, biocompatibility, and some portion of which provides optical clarity or the ability to be modified to allow optical clarity in the visual axis. The device of the present invention is compatible with IOL implants configured to be used in a natural capsule. The device of the present invention can be inserted into the eye via a small (<3.0 mm) incision in the eye wall. The device of the present invention is compatible with all known or conceivable ways of securing the device to the eye. Securement is preferably done with at least two-point securement to prevent IOL tilt and decentration. In some embodiments, the device may also include a proprietary or conventional method of securement.

To allow for expansion of the device as space permits, the device can also include a flexible ring structure. The device can include at least one aperture for the passage of light. The device of the present invention simplifies visual rehabilitation in cases of inadequate capsular support for IOL fixation. The present invention is compatible with existing or future laser or other procedures (e.g. Yag laser or surgical capsulotomy) for the treatment of secondary cataract (posterior capsule opacification).

In some instances, a patient may lack a capsule in which to place a lens in the eye. In other instances, a capsule may exist, but is too weak or damaged to support insertion of a lens. As a novel approach to IOL fixation in the absence of adequate capsular support, the present invention is directed to an implantable lens capsule that can be fixated to the globe which would be compatible with all existing or future IOLs designed for endocapsular implantation. This lens capsule would consist of an approximately 12.5-mm diameter pouch made from a thin, optically-clear, biocompatible, strong, and stable material, such as a biocompatible plastic. Alternatively, the material may not be optically clear but would be compatible with subsequent procedures such as YAG laser or surgical capsulotomy to create an aperture in the posterior aspect of the implanted lens capsule. The anterior aspect of the device would have an opening of sufficient diameter approximately 5 mm to allow for insertion of an IOL and to allow passage of light. The posterior aspect of the device could be either continuous, clear or non-clear, or could also have an aperture of sufficient size approximately 4 mm or smaller in diameter to prevent dislocation of an IOL into the posterior segment and to allow passage of light. The posterior aperture could be larger than 4 mm if the IOL haptic design ensured stability with such a larger opening. The device could contain a flexible, shape-retaining ring approximately 12.5-mm diameter to allow expansion of the device as space permits, similar to a capsular tension ring. At two or more points around the circumference of the lens capsule, single- or double-armed sutures composed of a durable, biocompatible material (e.g. Gore-Tex® polytetrafluoroethylene or other) would be attached for fixation to the sclera. These sutures would have needles, loops, or other features to facilitate passage through and/or attachment to the sclera. Other devices or material in addition to sutures could be attached to the lens capsule to enable fixation to the sclera.

The invention described herein has numerous advantages over current approaches to IOL fixation in the absence of native lens capsule support. The device is foldable and can be placed through a standard sized incision for cataract surgery. It is compatible with all IOL implants, including foldable one-piece lenses, designed for endocapsular fixation. Thus, no modification of the IOL would be needed for use with the device. The capsule material would be compatible with standard YAG laser, surgical, or other capsulotomy techniques to allow treatment in the event that the posterior capsule loses clarity after implantation. The capsule can be placed at a precise location posterior to the limbus to allow for predictable refraction after IOL implantation. The device could be easily removed from the eye through a small incision if needed.

The present invention includes an implantable lens capsule for intraocular lens insertion. The lens capsule is formed from a material which ideally would be optically clear. A non-clear material also could be used in which case the capsule could either contain a central posterior capsule aperture (−4 mm diameter) or be compatible with YAG, surgical, or other capsulotomy techniques. The anterior capsule includes a central aperture (−5 mm diameter) for insertion of the intraocular lens. The overall diameter of the lens capsule would be ˜12.5 mm and could include a flexible, shape-retaining ring of a similar diameter. Attached to the capsule would be material or devices such as single- or double-armed sutures of the appropriate composition to enable durable fixation of the capsule to the sclera. In the case of sutures, needles, loops, or other features could be incorporated to facilitate attachment to the sclera.

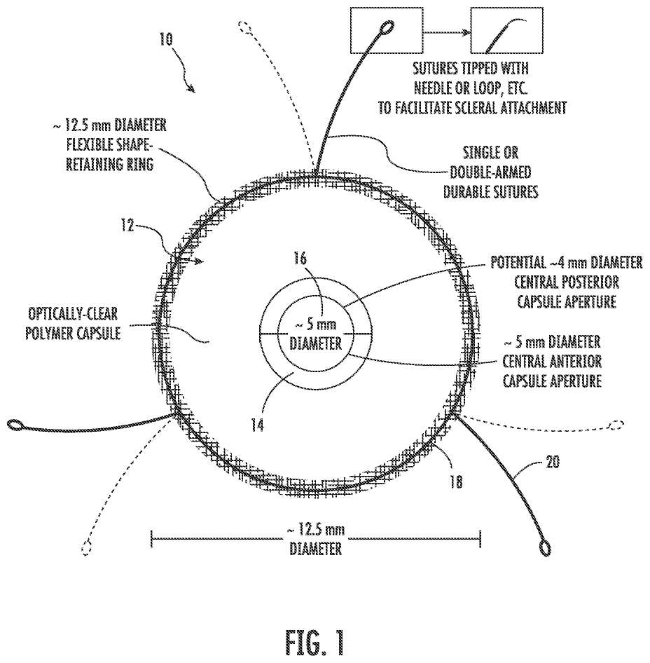

FIG. 1 illustrates a top down view of an implantable lens capsule device, according to an embodiment of the present invention. The implantable lens capsule device 10 includes a capsule 12. The capsule 12 includes a central anterior aperture 14. The central anterior aperture 14 is configured to allow the IOL to be inserted into the capsule 12. The central anterior aperture 14 can have a diameter of approximately 5 mm in order to allow for passage of the IOL. The capsule 12 includes an interior space defined by the outer material of the capsule 12. The interior space is configured to receive an IOL. The diameter of the outer circumference of the capsule is approximately 12.5 mm.

In some embodiments, the capsule 12 can be formed from an optically clear, biocompatible polymer material. The optically clear material allows for vision through the capsule. In other embodiments, the capsule 12 can be formed from an opaque material. In such an embodiment, the capsule includes a central posterior aperture 16 in order to allow light through the capsule to reach the retina. The central posterior aperture 16 can have a diameter of between approximately 2 and 4 mm in order to allow light in to the eye. The material used to form the present invention should be of a particular quality to allow the material to be cut by a laser, but also strong enough to hold the IOL in place. The capsule 12 is formed by fusing or adhering two sheets of polymer material together into the desired shape, by molding the polymer into the desired shape, or any other way of making the device that is known to or conceivable by one of skill in the art.

In some embodiments, the device 10 includes a retaining ring 18. The retaining ring 18 can be coupled to the capsule 12 by adhesive, fusion, or manufacturing the capsule 12 and the ring 18 as one piece. Alternately, the ring 18 can be physically separate from the capsule 12. The ring 18 is formed from a flexible material and can be a complete circle or can include a notch, gap, or other discontinuity, in order to allow for deformation. The notch gap or other discontinuity is not the only feature that allows for deformation, i.e. the ring material itself can be pliable/deformable, while still having enough shape memory to expand/resume/retain its shape after the deforming force (e.g. passing thru a small incision in the eye) ceases. The ring 18 allows the IOL to fully extend its arms. The diameter of the ring is approximately 12.5 mm.

The device 10 of the present invention can be fixed in the eye in any way known to or conceivable to one of skill in the art. One exemplary way to fix the device 10 in the eye is illustrated in FIG. 1. As illustrated in FIG. 1, the device 10 includes single or double armed sutures 20. The sutures can in some embodiments be tipped with a needle or a loop to facilitate scleral attachment. The sutures 20 can be positioned in three locations around the circumference of the device 10. Clamps or any other means of fixation can also be used.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A device for insertion into an eye, comprising:
a capsule having an outer surface defining an inner chamber configured to receive an intraocular lens (IOL), wherein the capsule defines an anterior aperture configured to allow the IOL to pass into the inner chamber and wherein the capsule defines a posterior aperture to allow for passage of light; and
an attachment mechanism for attaching the capsule to a sclera of the eye.

2. The device of claim 1 further comprising the capsule being formed from an optically clear material.

3. The device of claim 1 further comprising the capsule being formed from an opaque material.

4. The device of claim 1 further comprising forming the capsule from a polymer.

5. The device of claim 1 further comprising a flexible outer ring.

6. The device of claim 1 wherein the attachment mechanism comprises a suture.

7. The device of claim 6 wherein the suture comprises one selected from a group consisting of a needle tip and a loop tip.

8. The device of claim 6 further comprising a double armed suture.

9. The device of claim 1 wherein the posterior aperture measures between 2 mm and 4 mm.

10. The device of claim 5 further comprising one selected from a group consisting of a notch, discontinuity, or gap.

11. The device of claim 1 further comprising the device being formed from a material having strength, stability, and biocompatibility.

12. The device of claim 1 wherein the device is compatible with IOLs configured to be used in a natural capsule.

13. A method for providing a capsule for an intraocular lens (IOL) comprising:
creating a synthetic lens capsule wherein the synthetic lens capsule includes an outer surface defining an inner chamber configured to receive the IOL, and wherein the capsule defines an anterior aperture configured to allow the IOL to pass into the inner chamber and wherein the capsule defines a posterior aperture to allow for passage of light; and,
creating an attachment mechanism for attaching the capsule to a sclera of the eye.

14. The method of claim 13 further comprising surrounding the synthetic lens capsule with a flexible ring.

15. The method of claim 13 further comprising forming the synthetic lens capsule from a biocompatible plastic.

16. The method of claim 13 further comprising using a suture as the attachment mechanism.

17. The method of claim 16 further comprising ending the suture with a needle tip or a loop tip.

18. The method of claim 16 further comprising using a single arm or a double arm suture.

19. The method of claim 13 further comprising forming at least a portion of the capsule from an opaque material.

20. The method of claim 19 further comprising using an opaque material that is compatible with a YAG laser.
